SEQUENCE LISTING (3 sequences)

SEQ ID NO: 1 - DNA, 20 bp, Artificial Sequence
  Anti-sense origo nucleotide
gggtcagctgccaatgctag

SEQ ID NO: 2 - DNA, 4931 bp, Artificial Sequence
  pDNA sequence
gggcgaattggccaagtcggccgagctcgaattcgtcgacctcgaggcctcggaggattacaatagctaagaatttcgtcatcgctgaatacagttacattttacaatttggactttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgccacgcccgcgtcgaagatgttggggtgttgtaacaatatcgattccaattcagcgggggccacctgatatcctttgtatttaattaaagacttcaagcggtcaactatgaagaagtgttcgtcttcgtcccagtaagctatgtctccagaatgtagccatccatccttgtcaatcaaggcgttggtcgcttccggattgtttacataaccggacataatcataggtcctctgacacataattcgcctctctgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgtcgtatccctggaagatggaagcgttttgcaaccgcttccccgacttctttcgaaagaggtgcgcccccagaagcaatttcgtgtaaattagataaatcgtatttgtcaatcagagtgcttttggcgaagaatgaaaatagggttggtactagcaacgcactttgaattttgtaatcctgaagggatcgtaaaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctgacgcaggcagttctatgcggaagggccacacccttaggtaacccagtagatccagaggaattcattatcagtgcaattgttttgtcacgatcaaaggactctggtacaaaatcgtattcattaaaaccgggaggtagatgagatgtgacgaacgtgtacatcgactgaaatccctggtaatccgttttagaatccatgataataattttctggattattggtaattttttttgcacgttcaaaattttttgcaacccctttttggaaacaaacactacggtaggctgcgaaatgttcatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtattccgcgtacgtgatgttcacctcgatatgtgcatctgtaaaagcaattgttccaggaaccagggcgtatctcttcatagccttatgcagttgctctccagcggttccatcctctagaggatagaatggcgccgggcctttctttatgtttttggcgtcttccatttggatccgggccctctagatgcggccgcatgcataagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccgcaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 3 - RNA, 720 nt, Artificial Sequence
  EGFP mRNA
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaa

(19) United States
(12) Patent Application Publication
(10) Pub. No.: US 2021/0346290 A1
(43) Pub. Date: Nov. 11, 2021

(54) POLYION COMPLEX MICELLE THAT INCLUDES ANTISENSE OLIGONUCLEOTIDE AND BLOCK COPOLYMER OF PEG BLOCK AND CATIONIC POLYMER, SAID BLOCK COPOLYMER HAVING NUMBER-AVERAGE MOLECULAR WEIGHT OF 3-10 kDa

(71) Applicants: KAWASAKI INSTITUTE OF INDUSTRIAL PROMOTION, Kawasaki-shi (JP); THE UNIVERSITY OF TOKYO, Bunkyo-ku (JP)

(72) Inventors: Yasutaka ANRAKU, Kanagawa (JP); Kazunori KATAOKA, Kanagawa (JP); Shigeto FUKUSHIMA, Kanagawa (JP); Kanjiro MIYATA, Tokyo (JP); Hyun Su MIN, Tokyo (JP); Mitsuru NAITO, Tokyo (JP)

(73) Assignees: KAWASAKI INSTITUTE OF INDUSTRIAL PROMOTION, Kawasaki-shi (JP); THE UNIVERSITY OF TOKYO, Bunkyo-ku (JP)

(21) Appl. No.: 17/251,521

(22) PCT Filed: Jun. 13, 2019
(86) PCT No.: PCT/JP2019/023497

§ 371 Date: Jul. 12, 2021

(30) Foreign Application Priority Data
Jun. 13, 2018 (JP) 2018-112664

(51) Int. Cl. A61K 9/107; A61K 31/7088; A61K 47/34; A61K 9/00; A61P 3/08

(52) U.S. Cl. CPC A61K 9/107; A61K 31/7088; A61P 3/08; A61K 9/0019; A61K 47/34

(57) ABSTRACT

The present invention provides a polyion complex micelle including an antisense oligonucleotide and a block copolymer of a cationic polymer and a PEG block having a number average molecular weight of 3 kDa to 10 kDa.

### TECHNICAL FIELD

The present invention relates to a polyion complex micelle including an antisense oligonucleotide and a block copolymer of a PEG block having a number average molecular weight of 3 kDa to 10 kDa and a cationic polymer.

### BACKGROUND ART

Development of a drug delivery system is intended to efficiently and selectively deliver an encapsulated drug to a target tissue or organ. The drug delivery system can serve to enhance the retentivity in the blood of an encapsulated drug.

As a drug delivery system for the brain, Patent Literature 1 discloses a micelle having a surface modified with glucose. Patent Literature 1 reveals that when micelles each having a surface modified with glucose are administered while blood sugar manipulation is performed, accumulation of the micelles in the brain is markedly enhanced.

### CITATION LIST

### Patent Literature

Patent Literature 1: WO2015/075942

### SUMMARY OF INVENTION

The present invention provides a polyion complex micelle including an antisense oligonucleotide (or oligonucleotide) and a block copolymer of a PEG block having a number average molecular weight of 3 kDa to 10 kDa and a cationic polymer.

The present inventors have found that when an antisense oligonucleotide is mixed with a block copolymer of a PEG block having a number average molecular weight of 2 kDa and a cationic polymer, the formation efficiency of a polyion complex micelle is low. The present inventors have also found that when an antisense oligonucleotide is mixed with a block copolymer of a PEG block having a number average molecular weight of 12 kDa and a cationic polymer, a targeting molecule for drug delivery with which a polyion complex micelle is modified on the PEG block side enters inside the PEG, leading to a decrease in efficiency with which the targeting molecule is presented on the surface of the micelle. Further, the present inventors have found that a polyion complex micelle including nucleic acid and a block copolymer of a PEG block having a number average molecular weight of 3 kDa to 10 kDa and a cationic polymer can be formed with good efficiency when the nucleic acid is an antisense oligonucleotide, while the formation efficiency of the polyion complex micelle is low when the nucleic acid is DNA or mRNA, and that when the micelle is formed, a targeting molecule for drug delivery with which the micelle is modified on the PEG block side is easily exposed on the surface of the micelle.

According to the present invention, there are provided the following.

(1) A polyion complex micelle comprising an antisense oligonucleotide and a block copolymer of a PEG block having a number average molecular weight of 3 kDa to 10 kDa and a cationic polymer.
(2) The polyion complex micelle according to (1), wherein a PEG-side end of the block copolymer of a PEG block having a number average molecular weight of 3 kDa to 10 kDa and a cationic polymer, is modified with a molecule which binds to a cell surface or an extracellular matrix.
(3) The polyion complex micelle according to (2), wherein the molecule which binds to a cell surface is a GLUT1 ligand.
(4) The polyion complex micelle according to (3), wherein the GLUT1 ligand is glucose.
(5) The polyion complex micelle according to (4), wherein 10 to 40 mol % of the PEG-side end of the block copolymer of a PEG block having a number average molecular weight of 3 kDa to 10 kDa and a cationic polymer, is modified with glucose.
(6) The polyion complex micelle according to any one of (1) to (5), wherein the number average molecular weight of the PEG block is 4 kDa to 7 kDa.
(7) A pharmaceutical composition comprising the polyion complex micelle according to any one of (1) to (6).
(8) A pharmaceutical composition comprising the polyion complex micelle according to any one of (1) to (6) to be administered to a subject in accordance with a dosing regimen,

the dosing regimen including administering the pharmaceutical composition to a subject fasted or having hypoglycemia induced, and inducing elevation of blood sugar level in the subject,

the polyion complex micelle having an outer surface modified with a GLUT1 ligand.

### BRIEF DESCRIPTION OF DRAWINGS

FIG. 1A shows a relationship between the mixing ratio of a cationic polymer and an antisense oligo (N/P ratio) and the scattering light intensity (SLI) and the polydispersion index (PDI).

FIG. 1B shows a relationship between the ratio of the number of cationic polymers subjected to glucose modification to the total number of cationic polymers forming a micelle (glucose contents in micelle (%)) and the particle size and PDI.

FIG. 1C shows a relationship between the time for dispersion of ASO micelles in the presence of fetal bovine serum (FBS) and the concentration of ASO used for formation of the micelles.

FIG. 2 shows a fluorescence electron microscope image in which ASO micelles administered to a mouse is observed in the blood vessel of the ear.

FIG. 3 shows the blood retentivity of ASO micelles administered to a mouse (relative intensity).

FIG. 4A shows a relationship between the amount of ASO micelles accumulated in brain tissues of a mouse given fluorescently labeled ASO micelles and the ratio of glucose modification of the ASO surface, where the ordinate represents the ratio of the amount of micelles accumulated in the brain tissues to the dose, which is calculated in terms of a value per 1 g of brain tissues.

FIG. 4B shows a relationship between the amount of ASO micelles accumulated in brain tissues of an Alzheimer's disease mouse given fluorescently labeled ASO micelles and the ratio of glucose modification of the ASO surface, where the ordinate represents the ratio of the amount of micelles accumulated in the brain tissues to the dose, which is calculated in terms of a value per 1 g of brain tissues.

FIG. 5 shows the ratios of the accumulation of Glc-PIC (ASO) modified with glucose at 25% of the cationic polymer, in the brain and organs, to the accumulation of naked ASO in the brain and organs.

FIG. 6A shows the scattering light intensity (SLI) of an ASO micelle in which the average molecular weight of PEG in the PEG block is 2 kDa.

FIG. 6B shows the scattering light intensity (SLI) of an ASO micelle in which the average molecular weight of PEG in the PEG block is 12 kDa.

FIG. 6C shows the degrees of accumulation, in the mouse brain, of a model micelle in which the average molecular weight of PEG in the PEG block is 2 kDa and an ASO micelle in which the average molecular weight of PEG in the PEG block is 12 kDa.

FIG. 6D shows the formation efficiency of a micelle in which the average molecular weight of PEG in the PEG block is 5 kDa, and the anionic polymer is plasmid DNA (pDNA) or messenger RNA (mRNA), where the ordinate represents the scattering light intensity (SLI).

### DESCRIPTION OF EMBODIMENTS

In the present description, the term “micelle” means a vesicle formed of a layer of molecular membrane (or molecular layer). Examples of the micelle include micelles formed of amphipathic molecules such as surfactants, and micelles formed of polyion complexes (PIC micelles). It is known that preferably, the outer surface of the micelle is modified with polyethylene glycol from the viewpoint of blood retention time.

In the present description, the term “polyion complex” (hereinafter, also referred to as “PIC”) is an ion layer which is formed between a cationic block of a copolymer of PEG and the cationic block and an anionic block of a copolymer of PEG and the anionic block when the copolymers are mixed with each other in an aqueous solution so as to neutralize electric charge. The aim of linking PEG to the above-described charge chain is to inhibit the polyion complex from being aggregated and precipitated, and accordingly ensure that the polyion complex forms nanoparticles having monodisperse core-shell structures with a particle size of several tens of nanometers. Here, PEG covers the shells of the nanoparticles, and is therefore known to be advantageous in that high biocompatibility is obtained, and the blood retention time is improved. It is evident that in formation of the polyion complex, one of the charge block copolymers does not require a PEG part, and may be replaced by a homopolymer, a surfactant, nucleic acid and/or an enzyme. In formation of the polyion complex, at least one of the anionic polymer and the cationic polymer forms a copolymer with PEG, or both the polymers may form a copolymer with PEG. It is well known that PIC micelles are easily formed when the PEG content is increased, and PIC some is easily formed when the PEG content is decreased. Examples of the anionic polymer or block which is commonly used for preparation of the polyion complex include polyglutamic acid, polyaspartic acid and nucleic acid (e.g. DNA, mRNA and siRNA), and examples of the cationic polymer and block include polylysine and poly(5-aminopentylaspartic acid). Here, the mRNA means messenger RNA which is used for synthesis of protein by translation, and the siRNA means double-stranded RNA (nucleic acid) capable of inducing RNA interference (RNAi). The siRNA is not particularly limited, and is double-stranded RNA consisting of 20 to 30 bp, preferably 21 to 23 bp, 25 bp and 27 bp and having a sequence homologous with the sequence of a target gene. In the present invention, an antisense oligo can be used as an anionic polymer in PIC.

In the present description, the term “induce hypoglycemia” means that the blood sugar level is made lower than the blood sugar which would be presented unless a certain procedure were not carried out in the subject. Examples of the method for inducing hypoglycemia include administration of a diabetes drug. For example, in induction of hypoglycemia, the subject may take other drugs or drink a beverage such as water as long as the purpose of inducing hypoglycemia is achieved. Induction of hypoglycemia may be accompanied by other procedures having substantially no impact on the blood sugar.

In the present description, the term “fast” means that the subject is fasted for 3 hours or more, 4 hours or more, 5 hours or more, 6 hours or more, 7 hours or more, 8 hours or more, 9 hours or more, 10 hours or more, 11 hours or more, 12 hours or more, 13 hours or more, 14 hours or more, 15 hours or more, 16 hours or more, 17 hours or more, 18 hours or more, 19 hours or more, 20 hours or more, 21 hours or more, 22 hours or more, 23 hours or more, 24 hours or more, 25 hours or more, 26 hours or more, 27 hours or more, 28 hours or more, 29 hours or more, 30 hours or more, 31 hours or more, 32 hours or more, 33 hours or more, 34 hours or more, 35 hours or more, 36 hours or more, 37 hours or more, 38 hours or more, 39 hours or more, 40 hours or more, 41 hours or more, 42 hours or more, 43 hours or more, 44 hours or more, 45 hours or more, 46 hours or more, 47 hours or more, or 48 hours or more, for example. The fasting causes the subject to induce hypoglycemia. The fasting period is determined by a doctor etc. in light of the health condition of the subject, and is, for example, preferably a period equal to or greater than the amount of time for the subject to fall into a fasting blood sugar. For example, the fasting period may be equal to or greater than the amount of time taken until the expression of GLUT1 on the intravascular surfaces of cerebral vascular endothelial cells increases or reaches a plateau. The fasting period can be, for example, 12 hours or more, 24 hours or more or 36 hours or more among the above-described amounts of time. The fasting may be accompanied by other procedures having substantially no impact on the blood sugar level and the expression of GLUT1 on the intravascular surfaces.

In the present description, the term “inducing elevation of blood sugar level” means that the blood sugar level is elevated in a subject having hypoglycemia induced or a subject maintaining a hypoglycemic state. The blood sugar level can be elevated by various methods well known to those skilled in the art, and for example, the blood sugar level can be elevated by administration of a substance inducing elevation of blood sugar level, for example administration of a monosaccharide inducing elevation of blood sugar level, such as glucose, fructose (fruit sugar) or galactose or administration of a polysaccharide inducing elevation of blood sugar level, such as maltose, intake of a carbohydrate inducing elevation of blood sugar level, such as starch, or diet.

In the present description, the term “blood sugar manipulation” means that in a subject, hypoglycemia is induced, and the blood sugar level is then elevated. The blood sugar level of the subject can be maintained at a level of hypoglycemia after hypoglycemia is induced in the subject. The amount of time during which the blood sugar level of the subject is maintained at a level of hypoglycemia can be, for example, 0 hours or more, an hour or more, 2 hour or more, 3 hours or more, 4 hours or more, 5 hours or more, 6 hours or more, 7 hours or more, 8 hours or more, 9 hours or more, 10 hours or more, 11 hours or more, 12 hours or more, 13 hours or more, 14 hours or more, 15 hours or more, 16 hours or more, 17 hours or more, 18 hours or more, 19 hours or more, 20 hours or more, 21 hours or more, 22 hours or more, 23 hours or more, 24 hours or more, 25 hours or more, 26 hours or more, 27 hours or more, 28 hours or more, 29 hours or more, 30 hours or more, 31 hours or more, 32 hours or more, 33 hours or more, 34 hours or more, 35 hours or more, 36 hours or more, 37 hours or more, 38 hours or more, 39 hours or more, 40 hours or more, 41 hours or more, 42 hours or more, 43 hours or more, 44 hours or more, 45 hours or more, 46 hours or more, 47 hours or more, or 48 hours or more. Thereafter, the blood sugar level can be elevated. In the present description, the term “maintaining the blood sugar” indicates that the subject may take, for example, other drugs or drink a beverage such as water as long as the purpose of maintaining hypoglycemia in the subject is achieved. Induction of hypoglycemia may be accompanied by other procedures having substantially no impact on the blood sugar.

In the present description, the term “subject” is a mammal such as a human. The subject may be a healthy subject or a subject with some disease. Here, examples of the disease include cranial nerve diseases, for example psychotic disorders, depression, mood disorders, anxiety, sleep disorders, dementia and substance-related disorders. The dementia is not particularly limited, and examples thereof include Alzheimer's disease and Creutzfeldt-Jacob disease.

In the present description, the term “blood-brain barrier” is a functional barrier existing between the blood flow and the brain and having selectivity on permeation of substances. It is considered that the blood-brain barrier is constituted by cerebral vascular endothelial cells and the like. While much of the substance permeability of the blood-brain barrier is unknown, glucose, alcohol and oxygen are known to easily pass through the blood-brain barrier, and it is considered that lipid-soluble substances and small molecules (with a molecular weight of, for example, less than 500) tend to more easily pass through the blood-brain barrier than water-soluble molecules and macromolecules (with a molecular weight of, for example, 500 or more). Many of brain disease treatment agents and brain diagnostic agents are unable to pass through the blood-brain barrier, which is a major obstacle to treatment of brain disease, analysis of the brain, and the like. In the present description, the term “blood-nerve barrier” is a functional barrier existing between the blood flow and the peripheral nerve and having selectivity on permeation of substances. In the present description, the term “blood-spinal fluid barrier” is a functional barrier existing between the blood flow and the cerebral spinal fluid and having selectivity on permeation of substances. In the present description, the term “blood-retina barrier” is a functional barrier existing between the blood flow and the retina tissues and having selectivity on permeation of substances. It is considered that the blood-nerve barrier, the blood-spinal fluid barrier and the blood-retina barrier are constituted by vascular endothelial cells and the like existing in the barriers, and are similar in function to the blood-brain barrier.

In the present description, the term “GLUT1 ligand” means a substance which specifically binds to GLUT1. As the GLUT1 ligand, various ligands are known, and there is no particular limitation. Examples thereof include molecules such as glucose and hexose, and the GLUT1 ligand can be used instead of glucose for preparation of carriers and conjugates in the present invention. The GLUT1 ligand is preferably comparable to or higher than glucose in affinity with GLUT1. 2-N-4-(1-azi-2,2,2-trifluoroethyl)benzoyl-1,3-bis(D-mannos-4-yloxy)-2-propylamine (ATB-BMPA), 6-(N-(7-nitrobenz-2-oxa-1,3-diazol-4-yl)amino)-2-deoxyglucose (6-NBDG), 4,6-O-ethylidene-α-D-glucose, 2-deoxy-D-glucose and 3-O-methylglucose are known to bind to GLUT1, and these molecules can also be used as the GLUT1 ligand in the present invention.

In the present description, the terms “antisense oligonucleotide”, “antisense oligo”, “ASO” and “antisense compound” are used interchangeably, and each refer to a molecule which can hybridize with a target nucleic acid produced from cells under physiological conditions (e.g. in cells). The “antisense oligo” or the “antisense compound” is a molecule which is polymer of monomer units, where each monomer unit has a base and a backbone, and the backbone has a linkage between monomers, thereby enabling Watson-Click-type base pairing between the antisense oligo and a target nucleic acid (e.g. RNA). As the antisense oligo, for example, nucleic acids, particularly various nucleic acids such as RNA, DNA, and their analogues with higher stability (e.g. locked nucleic acids (LNA), bridged nucleic acids (BNA), morpholino oligos and peptide nucleic acids) have been developed. The antisense oligos all have a base for formation of Watson-Click-type base pairing with a target nucleic acid, and are anionic polymers. In an embodiment, the antisense oligo (or oligonucleotide) can have a length of 15 bases or more, a length of 16 bases or more, a length of 17 bases or more, a length of 18 bases or more, a length of 19 bases or more, a length of 20 bases or more, a length of 21 bases or more, a length of 22 bases or more, a length of 23 bases or more, a length of 24 bases or more, a length of 25 bases or more or a length of 26 bases or more, and/or a length of 100 bases or less, a length of 90 bases or less, a length of 80 bases or less, a length of 70 bases or less, a length of 60 bases or less, a length of 50 bases or less, a length of 40 bases or less or a length of 30 bases or less. In an embodiment, the antisense oligo (or oligonucleotide) can have a length of 15 to 50 bases, a length of 15 to 40 bases, a length of 15 to 30 bases, a length of 20 to 50 bases, a length of 20 to 40 bases, a length of 20 to 30 bases, a length of 18 to 40 bases, a length of 18 to 30 bases, a length of 18 to 25 bases or a length of 21 to 26 bases. In an embodiment, the antisense oligo can be a nucleic acid such as RNA or DNA in which nucleic acid analogues with higher stability are present at both ends.

In the present description, the term “monodisperse” means that the particle group is a particle group in which the standard deviation of particle sizes of the particles is within 10%. Here, the particle size can be determined, for example, in accordance with a dynamic scattering light method. An autocorrelation function is determined by a photon correlation method from temporal fluctuations of observed scattering light, and a diffusion coefficient, a particle size and a particle size distribution are determined by cumulant method and histogram method analysis, etc.

The present inventors have shown that when the number average molecular weight of PEG forming a PEG block is small in a PIC micelle of an antisense oligo and a block copolymer of the PEG block and a cationic polymer block, the micelle formation efficiency decreases. The present inventors have also shown that when the number average molecular weight of PEG forming a PEG block is large in a PIC micelle of an antisense oligo and a block copolymer of the PEG block and a cationic polymer block, a targeting molecule for drug delivery is hardly expressed on the micelle surface although the micelle is modified with the targeting molecule on the PEG block side. Further, the present inventors have found that when the number average molecular weight of PEG forming a PEG block is moderate in a PIC micelle with an antisense oligo, excellent micelle formation efficiency is obtained, and a targeting molecule is easily exposed on the micelle surface, leading to completion of the present invention. That is, according to the present invention, there is provided a PIC micelle of an antisense oligo and a block copolymer of a PEG block and a cationic polymer block, the PEG block having a number average molecular weight of, for example, 3 kDa to 10 kDa.

In the present invention, the number average molecular weight of a PEG block in the block copolymer of the PEG block and a cationic polymer block is not particularly limited, and for example, the lower limit thereof can be 3 kDa or more, 3.5 kDa or more, 4 kDa or more, or 4.5 kDa or more. In the present invention, the upper limit of the number average molecular weight of a PEG block in the block copolymer of the PEG block and a cationic polymer block can be 11 kDa or less, 10 kDa or less, 9 kDa or less, 8 kDa or less, 7 kDa or less, 6 kDa or less, or 5.5 kDa or less. In the present invention, for example, the number average molecular weight of a PEG block in the block copolymer of the PEG block and a cationic polymer block can be a value in a range between one lower limit selected from the above-described lower limits and one upper limit selected from the above-described upper limits. In the present invention, the number average molecular weight of a PEG block in the block copolymer of the PEG block and a cationic polymer block can be, for example, 3 kDa to 10 kDa, 3.5 kDa to 9 kDa, 4 kDa to 7 kDa, 4 kDa to 6 kDa, for example about 5 kDa (e.g. 5 kDa±30%, ±25%, ±20%, ±15% or ±10%).

According to the present invention, the antisense oligo which is anionic can form a polyion complex micelle with the block copolymer of a PEG block and a cationic polymer block by ionic interaction. In the polyion complex micelle of the block copolymer and the antisense oligo which is anionic, the antisense oligo optionally has a modification with PEG. In an embodiment, the antisense oligo may have a PEG block having a number average molecular weight equal to or less than 5%, equal to or less than 10%, equal to or less than 15%, equal to or less than 20%, equal to or less than 30%, equal to or less than 40%, equal to or less than 50%, equal to or less than 60%, equal to or less than 70%, equal to or less than 80%, equal to or less than 90% or equal to or less than 95% of the number average molecular weight of a PEG chain of the block copolymer. In an embodiment, the antisense oligo does not a modification with PEG.

The mixing ratio of the antisense oligo and the block copolymer of a PEG block and a cationic polymer block can be, for example, 1.0 to 2.0, 1.1 to 1.9, 1.2 to 1.8, 1.3 to 1.7, 1.4 to 1.6 or about 1.5 in terms of a N/P ratio (ratio of the number of nitrogen atoms of the polymer side chain and the number of phosphate groups of DNA).

In the present description, PEG can be linear PEG. In the present invention, another hydrophilic polymer block may be used in place of PEG.

In the present invention, a polymer of cationic monomer units can be used as the cationic polymer. The cationic monomer unit is not particularly limited, and for example, an unnatural cationic amino acid (e.g. modified natural cationic amino acid), or a natural cationic amino acid can be used as a cationic monomer unit. Examples of the natural cationic amino acid include one or more natural cationic amino acids selected from lysin and ornithine. Examples of the unnatural cationic amino acid include one or more modified natural amino acids selected from glutamic acid and aspartic acid modified with a cationic side chain. Examples of the cationic side chain include groups having a 5-aminopentyl group, a 4-aminobutyl group, a 3-aminopropyl group and a 2-aminoethyl group, and in these groups, C may be replaced by N. Examples of the cationic side chain include —NH—CH₂CH₂—NH₂, —NH—CH₂CH₂—NH—CH₂CH₂—NH₂ (sometimes referred to as diethyltriamine (DET)), —NH—CH₂CH₂—NH—CH₂CH₂—NH—CH₂CH₂—NH₂ (sometimes referred to as triethyltetraamine (TET)) and —NH—CH₂CH₂—NH—CH₂CH₂—NH—CH₂CH₂—NH—CH₂CH₂—NH₂ (sometimes referred to as tetraethylpentaamine (TEP)). For example, for improving the convenience of crosslinking, the side chain of the cationic amino acid may be modified with 2-iminothiolane or dimethyl 3,3′-dithiobis(propionimidate) (DTBP) {at least some or all of amino acids are still cationic}.

In the present invention, the PEG-side end of the block copolymer of a PEG block and a cationic polymer block may be modified with a targeting molecule for drug delivery. Here, the targeting molecule for drug delivery is a molecule (e.g. antibody or antigen-binding fragment of antibody, ligand of receptor, lectin, or peptide having affinity, such as RGD peptide or cRGD peptide) which selectively binds to a molecule (e.g. membrane protein, sugar protein, receptor or the like) that is exposed on micelle surfaces and selectively expressed on the surface of an internal body site to be targeted when micelles are formed using the block copolymer. In the present description, the term “selective” means being bindable to a specific molecule with stronger affinity than to other molecules. In drug delivery, a targeting molecule which selectively binds to a molecule that is selectively expressed on the surface of an internal body site to be targeted is exposed on micelle surfaces to accumulate micelles on the internal body site to be targeted.

In the present description, examples of the targeting molecule for drug delivery include molecules binding to cell surfaces, or molecules binding to extracellular matrixes. The molecule binding to cell surfaces can be, for example, a receptor exposed on cell surfaces, membrane protein such as a channel, or a molecule binding to sugar protein. For example, GLUT1 is expressed on surfaces of vascular endothelial cells on the blood vessel side at a specific position in the body. Thus, examples of the targeting molecule for drug delivery include molecules binding to GLUT1 (hereinafter, referred to as “GLUT1 ligands”). As the GLUT1 ligand, various ligands are known. The GLUT1 ligand is not particularly limited, and examples thereof include molecules of glucose, hexose and the like. Examples of the GLUT1 ligand include 2-N-4-(1-azi-2,2,2-trifluoroethyl)benzoyl-1,3-bis(D-mannos-4-yloxy)-2-propylamine (ATB-BMPA), 6-(N-(7-nitrobenz-2-oxa-1,3-diazol-4-yl)amino)-2-deoxyglucose (6-NBDG), 4,6-O-ethylidene-α-D-glucose, 2-deoxy-D-glucose and 3-O-methylglucose which are known to bind to GLUT1.

The antisense oligo which can be used in the present invention is not particularly limited, and examples thereof include antisense oligos targeting a gene selected from the group consisting of a BACE1 gene, a RAGE gene, a Marat1 gene, a VII factor, Eg5, PCSK9, TPX2, apoB, SAA, TTR, RSV, a PDGF beta gene, an Erb-B gene, a Src gene, a CRK gene, a GRB2 gene, a RAS gene, a MEKK gene, a JNK gene, a RAF gene, an Erkl/2 gene, a PCNA (p21) gene, a MYB gene, a JUN gene, a FOS gene, a BCL-2 gene, a cyclin D gene, a VEGF gene, an EGFR gene, a cyclin A gene, a cyclin E gene, a WNT-1 gene, a beta-catenin gene, a c-MET gene, a PKC gene, a NFKB gene, a STAR3 gene, a survivin gene, a Her2/Neu gene, a SORT1 gene, a XBP1 gene, a topoisomerase I gene, a topoisomerase II alpha gene, a p73 gene, a p21 (WAF1/CIP1) gene, a p27 (KIP1) gene, a PPM1D gene, a RAS gene, a caveolin I gene, MIB I gene, MTAI gene, a M68 gene, a cancer suppressor gene and a p53 cancer suppressor gene. For example, the antisense oligo can target genes encoding development-related proteins (e.g. adhesion molecules, cyclin kinase inhibitors, Wnt family members, Pax family members, Winged helix family members, Hox family members, cytokine/lymphokine and receptors thereof, growth/differentiation factors and receptors thereof, or neurotransmitters and receptors thereof); proteins encoding tumor genes (e.g. ABLI, BCLI, BCL2, BCL6, CBFA2, CBL, CSFIR, ERBA, ERBB, EBRB2, ERBB2, ERBB3, ETSI, ETSI, ETV6, FGR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3 or YES); tumor suppressor proteins (e.g. APC, BRCA1, BRCA2, MADH4, MCC, NF1, NF2, RBl, TP53 or WTI); and enzymes (e.g. ACC synthases and oxidases, ACP desaturases and hydroxylases, ADP glucose pyrophosphorylases, acetylases and deacetylases, ATPases, alcohol dehydrogenases, amylases, amyloglucosidases, catalases, cellulases, chalcone synthases, kinases, cyclooxygenases, decarboxylases, dextrinases, DNA and RNA polymerases, galactosidases, glucanases, glucose oxidases, granule-bound starch synthetases, GTPases, helicases, hemicellulases, integrases, inulinases, invertases, isomerases, kinases, lactases, lipases, lipoxygenases, lysozymes, nopaline synthetases, octopine synthetases, pectinesterases, peroxidases, phosphatases, phospholipases, phosphorylases, phytases, plant growth regulation factor synthetases, polygalacturonases, proteinases and peptidases, pullulanases, recombinases, reverse transcriptases, RUBISCO, topoisomerases or xylanases). For example, the antisense oligo can target genes encoding proteins involved in tumor growth (including angiogenesis) or metastatic activity. For example, the antisense oligo can target genes encoding one or more secretory proteins, cell cycle regulatory proteins, gene regulatory proteins, apoptosis regulatory proteins, or proteins involved in immune response, inflammation, complement cascades or blood coagulation systems. For example, the antisense oligo can target genes encoding c-myc, c-myb, mdm2, PKA-I (type I protein kinase A), Ras, c-Raf kinases, CDCl25 phosphatase, cyclin, cyclin dependent kinases (cdks), telomerases, PDGF/sis and mos. For example, the antisense oligo can be used for targeting mRNA encoded by a fusion gene developed chromosomal translocation, such as a Bcr/Abl fusion tumor gene. For example, the antisense oligo can target genes encoding proteins such as cyclin dependent kinases, proliferating cell nuclear antigens (PCNA), transforming growth factors β (TGF-β), nuclear factors κB (NF-κB), E2F, HER-2/neu, PKA, TGF-α, EGFR, TGF-β, IGFIR, P12, MDM2, VEGF, MDR, transferrin, ferritin, ferritin receptors, transferrin receptors, IRE, C-fos, HSP27 and metallothionein.

According to the present invention, the PIC micelle can be prepared by mixing an antisense oligo with a block copolymer of a cationic polymer block and a PEG block having a number average molecular weight of 3 kDa to 10 kDa. Here, the block copolymer of a cationic polymer block and a PEG block having a number average molecular weight of 3 kDa to 10 kDa may be mixed with the antisense oligo at a quantitative ratio which ensures electric neutralization. The PIC micelle may be formed, followed by crosslinking polymers forming the micelle. The crosslinking can be performed using, for example, a crosslinker. The crosslinking may be performed by S—S bonding of 2-iminothiolane and dimethyl 3,3′-dithiobis(propionimidate) (DTBP) introduced to the side chain of the cationic polymer as disclosed in Examples of the present invention. In the present invention, it is considered that theoretically, the targeting molecule for drug delivery is expressed on micelle surfaces by regulating the number average molecular weight of PEG even when the PIC micelle is free of crosslinking.

In the present invention, the block copolymer of a PEG block having a number average molecular weight of 3 kDa to 10 kDa and a cationic polymer block, and the antisense oligo form an ion layer between the block copolymer and the antisense oligo, and in this way, a polyion complex micelle can be formed. In an embodiment of the present invention, the PEG-side end of the block copolymer of a PEG block having a number average molecular weight of 3 kDa to 10 kDa and a cationic polymer block may be modified with, for example, a GLUT1 ligand such as glucose. In an embodiment of the present invention, glucose may modify the PEG block via an oxygen atom connected to carbon at the 6-position of the glucose. In an embodiment of the present invention, the GLUT1 ligand is connected to the PEG end of the cationic polymer.

In the present invention, the PIC micelle including an antisense oligo and a GLUT1 ligand-modified block copolymer of a cationic polymer block and a PEG block having a number average molecular weight of 3 kDa to 10 kDa can be administered in accordance with a dosing regimen, and the dosing regimen includes administering the micelle to a subject fasted or having hypoglycemia induced, and inducing elevation of blood sugar level in the subject. WO2015075942A indicates that when administered, vesicles each having a surface modified with a GLUT1 ligand are delivered to the brain with higher efficiency than vesicles whose surfaces are not modified with a GLUT1 ligand. WO2015075942A indicates that when administered in accordance with the dosing regimen, vesicles each having a surface modified with a GLUT1 ligand are delivered to the brain with further higher efficiency than vesicles which are merely administered without undergoing the above-mentioned manipulation and have a surface modified with a GLUT1 ligand. The PIC micelle including an antisense oligo and a GLUT1 ligand-modified block copolymer of a PEG block having a number average molecular weight of 3 kDa to 10 kDa and a cationic polymer block can pass through the blood-nerve barrier, the blood-retina barrier or the blood-spinal fluid barrier.

In the present invention, the PIC micelle including an antisense oligo and a GLUT1 ligand-modified block copolymer of a PEG block having a number average molecular weight of 3 kDa to 10 kDa and a cationic polymer may further include a GLUT1 ligand-free block copolymer of a PEG block having a number average molecular weight of 3 kDa to 10 kDa and a cationic polymer. In an embodiment of the present invention, the GLUT1 ligand-modified block copolymer of a PEG block having a number average molecular weight of 3 kDa to 10 kDa and a cationic polymer and the GLUT1 ligand-free block copolymer of a cationic polymer block and a PEG block having a number average molecular weight of 3 kDa to 10 kDa may be contained in the PIC micelle at a molar ratio of 10:90 to 40:60 (e.g. 20:80 to 30:70, for example 25:75). Alternatively, the PEG-side end of the block copolymer of a PEG block having a number average molecular weight of 3 kDa to 10 kDa and a cationic polymer may be modified with glucose at 10 to 40 mol % (e.g. 20 to 20 mol %, for example 25 mol %). When the ratio of the block copolymer modified with a GLUT1 ligand is 10:90 to 40:60 (or 10 to 40 mol %), the PIC micelle easily passes through vascular endothelial cells, and is easily accumulated in a larger amount in tissue parenchyma. In an embodiment of the present invention, the GLUT1 ligand-modified block copolymer of a PEG block having a number average molecular weight of 3 kDa to 10 kDa and a cationic polymer and the GLUT1 ligand-free block copolymer of a PEG block having a number average molecular weight of 3 kDa to 10 kDa and a cationic polymer may be contained in the PIC micelle at a molar ratio of 50:50 to 100:0 (e.g. 60:40 to 90:10 or 70:30 to 80:20). Alternatively, the PEG-side end of the block copolymer of a cationic polymer and a PEG block having a number average molecular weight of 3 kDa to 10 kDa may be modified with glucose at 50 to 100 mol %. It is considered that when the ratio of the block copolymer modified with a GLUT1 ligand is 50:50 to 100:0 (or 50 mol % to 100 mol %), the PIC micelle is incorporated into vascular endothelial cells, more hardly released into tissue parenchyma from the vascular endothelial cells, and resultantly easily accumulated in the vascular endothelial cells. Therefore, the ratio of the block copolymer modified with GLUT1 ligand in PIC can be adjusted depending on whether the PIC micelle is to be allowed to pass through vascular endothelial cells expressing GLUT1 or to be accumulated in vascular endothelial cells expressing GLUT1.

The micelle of the present invention can be administered by either oral administration or parenteral administration (e.g. intravenous administration or intraperitoneal administration).

According to the present invention, there is provided a pharmaceutical composition containing the micelle of the present invention. The pharmaceutical composition of the present invention may contain the micelle of the present invention and a pharmaceutically acceptable excipient.

### EXAMPLES

### Example 1: Construction of PIC (ASO) Micelle

In this Example, a polyion complex (PIC) micelle (hereinafter, sometimes referred to as a “PIC (ASO) micelle”) was constructed using an antisense oligo (ASO) and a cationic polymer having a polyethylene glycol (PEG) block. For examining accumulation efficiency in the brain, Glc(6) was connected to a PEG chain in the same manner as described above. For introducing intermolecular crosslinking without using a crosslinker, 2-iminothiolane (2-IT) or dimethyl 3,3′-dithiobis(propionimidate) (DTBP) was introduced to a lysin side chain. The length of PEG was set to a number average molecular weight of 5 kDa.

### (1) Synthesis of DIG(6)

11.7 g of 1,2-α-isopropylidene-α-D-glucofuranose (manufactured by Tokyo Chemical Industry Co., Ltd.) was weighed and taken into an eggplant flask, 60 mL of pyridine (manufactured by Wako Pure Chemical Industries, Ltd.) was added thereto, the mixture was heated with a dryer to be dissolved, and 60 mL of dichloromethane (manufactured by Wako Pure Chemical Industries, Ltd.) was then added. 7.2 mL of pivaloyl chloride (manufactured by Wako Pure Chemical Industries, Ltd.) was added dropwise, and the mixture was stirred and mixed at room temperature for 5 hours. After the reaction, the solvent was removed under reduced pressure, and 100 mL of pure water was added with mild stirring. A white solid material precipitated was recovered by suction filtration (Kiriyama Paper Filter 5C), and washed with pure water. The obtained white solid material was dried under reduced pressure. All the dried white solid material was dissolved in a small amount of methanol heated to 65° C., and was then cooled to be purified by recrystallization. In this way, an acicular crystal was obtained.

290 mL of dimethoxypropane (manufactured by Wako Pure Chemical Industries, Ltd.) and 367 mg of p-toluenesulfonic acid (manufactured by Wako Pure Chemical Industries, Ltd.) were added, the mixture was heated to 75° C. to be dissolved, the solution was added to the acicular crystal, and the mixture was stirred and mixed at 75° C. for 30 minutes, and turned back to room temperature. After the reaction, 1 mL of triethylamine (manufactured by Wako Pure Chemical Industries, Ltd.) was added dropwise little by little, and 50 mL of toluene (manufactured by Wako Pure Chemical Industries, Ltd.) was added. The mixed solution was concentrated to about 50 mL under reduced pressure, 50 mL of toluene was added again, and the mixture was concentrated to about 20 mL again. The obtained concentrate was extracted three times with 100 mL of dichloromethane and 100 mL of pure water, the dichloromethane layer was recovered in a beaker, an appropriate amount of anhydrous sodium sulfate (manufactured by Wako Pure Chemical Industries, Ltd.) was added, and the mixture was stirred and mixed for 30 minutes. The solid material was removed by suction filtration (Kiriyama Paper Filter 5C), and the filtrate was concentrated under reduced pressure. The concentrated oily solution was subjected to separation and purification with silica gel (manufactured by Merck Company). Hexane (manufactured by Wako Pure Chemical Industries, Ltd.):ethyl acetate (manufactured by Wako Pure Chemical Industries, Ltd.)=7:3 was used as a developing solvent.

30 mL of methanol (manufactured by Wako Pure Chemical Industries, Ltd.) was added to the purified product, and the mixture was heated to 70° C. to be dissolved. 60 mL of a 5 N sodium hydroxide aqueous solution was added, and the mixture was vigorously stirred at 70° C. for 1 hour. 300 mL of dichloromethane was added to the reaction liquid for extraction, the dichloromethane layer was recovered, and the extraction was then repeated twice under the same conditions. An appropriate amount of anhydrous sodium sulfate was added to the extract, the mixture was stirred for 30 minutes, the solid material was removed by suction filtration (Kiriyama Paper Filter 5C), and dichloromethane was concentrated under reduced pressure to obtain a white solid material. The white solid material was dissolved in a small amount of ethanol heated to 75° C., the solution was cooled, and a granular crystal precipitated was recovered by suction filtration (Kiriyama Paper Filter 5C), and dried under reduced pressure to obtain DIG(6).

### (2) Synthesis of DIG(6)-PEG-NH₂

520 mg of DIG(6) was weighed and taken, and dissolved in a small amount of dichloromethane and benzene, and the solution was freeze-dried. 75 mL of tetrahydrofuran purified by distillation in an argon atmosphere was added to the dried DIG(6), 6.7 mL of a potassium naphthalene solution was added at 0.3 mol/L, and the mixture was stirred for 5 minutes. To this was added 13.5 mL of ethylene oxide purified by distillation, and the mixture was stirred at 25° C. for 3 days. After 3 days, the reaction solution was added dropwise to 1.5 L of diethyl ether (manufactured by Showa-Ether), and a precipitate was recovered by suction filtration (JCWP 10 μm) to obtain DIG(6)-PEG-OH.

6 g of DIG(6)-PEG-OH was weighed and taken into a flask, dissolved in benzene, and then freeze-dried. 40 mL of tetrahydrofuran purified by distillation in an argon atmosphere was then added, and 0.32 mL of triethylamine purified by distillation was added. 20 mL of tetrahydrofuran purified by distillation was taken into another flask, and 0.19 mL of methanesulfonic acid chloride purified by distillation was added. The PEG solution was slowly added dropwise while the solution of methanesulfonic acid chloride was cooled in a water bath, and the mixture was stirred for 2 hours. A precipitate generated during the reaction was removed by suction filtration (JCWP 10 μm), the filtrate was added dropwise to 1 L of diethyl ether, and a precipitate was recovered by suction filtration (Kiriyama Paper Filter 5C). Thereafter, the precipitate was put into an eggplant flask, and dissolved in 500 mL of aqueous ammonia (manufactured by Wako Pure Chemical Industries, Ltd.), and the solution was stirred at room temperature for 4 days. Thereafter, the reaction liquid was concentrated under reduced pressure, and purified with a cation-exchange column to obtain DIG(6)-PEG-NH₂ (molecular weight 5,300).

### (3) Synthesis of Glc(6)-PEG-PLys

530 mg of DIG(6)-PEG-NH₂ was weighed and taken into a flask, and dissolved by adding benzene, and the solution was freeze-dried. Thereafter, the dried product was dissolved by adding 12 mL of N,N-dimethylformamide purified by distillation, in which thiourea (manufactured by Wako Pure Chemical Industries, Ltd.) was dissolved at a concentration of 1 mol/L. 1,330 mg of NCA-Lys (TFA) was weighed and taken into another flask in an argon atmosphere, and dissolved by adding 26 mL of N,N-dimethylformamide purified by distillation, in which thiourea was dissolved at a concentration of 1 mol/L. NCA-Lys (TFA) was added to the PEG solution, and the mixture was stirred and mixed at 25° C. for 3 days. The reaction liquid was injected into a dialysis membrane (MWCO: 3,500) to dialyze it with water as an external liquid, and DIG(6)-PEG-PLys (TFA) was recovered by freeze-drying.

After the drying, 500 mg of DIG(6)-PEG-PLys was taken into a flask, and dissolved by adding 50 mL methanol and 5 mL of 1 N sodium hydroxide, and the solution was stirred at 35° C. for 24 hours. Thereafter, the reaction liquid was injected into a dialysis membrane (MWCO: 3,500) to dialyze it with water as an external liquid, and DIG(6)-PEG-PLys was recovered by freeze-drying.

The obtained DIG(6)-PEG-PLys was dissolved in 80% trifluoroacetic acid (manufactured by Tokyo Chemical Industry Co., Ltd.), the solution was reacted at room temperature for 30 minutes, and the reaction liquid was neutralized with sodium hydroxide, and then injected into a dialysis membrane (MWCO: 3,500) to be dialyzed. The dialysis was performed three times with 0.01 N hydrochronic acid as an external liquid, and three times with pure water as an external liquid, and Glc(6)-PEG-PLys was recovered by freeze-drying. The number average polymerization degree of lysin was 42.

Thereafter, 2-IT or DTBP was introduced to the side chain of lysin in accordance with the following scheme.

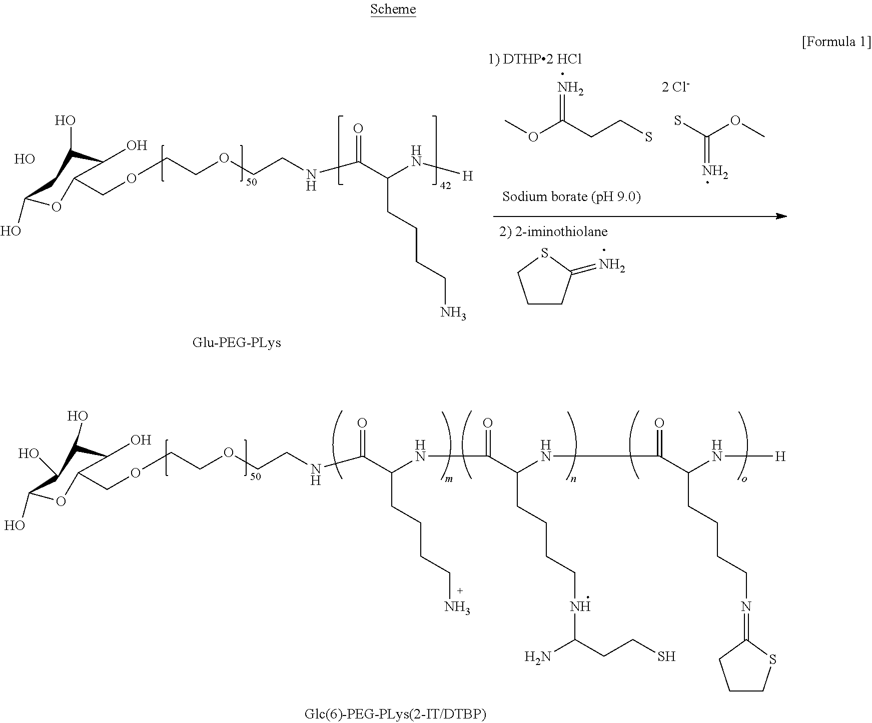

50 mg of the Glc(6)-PEG-PLys thus obtained was dissolved in 5 mL of a 0.1 M borax buffer solution (pH 9.0). In another vessel, 5.5. mg of di-tert-butyl peroxide (DTBP manufactured by Thermo-Fisher Scientific) (0.1 equivalent to PLys of Glc(6)-PEG-PLys) was dissolved in cold water. The DTBP solution was added dropwise to the Glc(6)-PEG-PLys solution, and the mixture was stirred at 25° C. for 45 minutes. Thereafter, 58 mg of 2-iminothiolane (2-IM) (manufactured by Thermo-Fisher Scientific) (2.4 equivalent to PLys of Glc(6)-PEG-PLys) was added in an original powder form, and the mixture was stirred at 25° C. for 30 minutes. The reaction liquid was heated to 35° C., and stirred overnight. The reaction solution was transferred into a dialysis membrane (MWCO: 3,500), and dialyzed with a 10 mM phosphate buffer solution (pH 7.4) (the external solution was exchanged in 30 minutes; dialyzed twice). 54 mg of DTT (manufactured by Wako Pure Chemical Industries, Ltd.) (2.0 equivalent to PLys of Glc(6)-PEG-PLys) was then added to the internal solution of the dialysis membrane, and the mixture was left standing for 30 minutes, and then dialyzed with 150 mM NaCl solution for 30 minutes three times, and with pure water for 30 minutes three times. After the dialysis, the dialysate was treated with a filter (Sterivex (trademark) GP 0.22 μm manufactured by Nihon Millipore K.K.), and then freeze-dried to obtain white powder of Glc(6)-PEG-PLys (DTBP/IM) (yield: 60.4 mg). The amounts of DTBP and IM bound to PEG-PLys (DTBP/IM) were calculated from the CH₂ peak of DTBP measured by ¹H-NMR, and the ratio of the integral values of peaks of main chain of CH₂ of IM and CH₂ of PEG. The introduction ratios of DTBP and IM of the obtained Glc(6)-PEG-PLys (DTBP/IM) were 18% and 82%, respectively (that is, a molecule of the above formula was obtained, where a is 0.2, b is 6.2, c is 35.5 and a+b+c is 41.9).

### (3) Preparation of Glc-PIC (ASO) Micelle

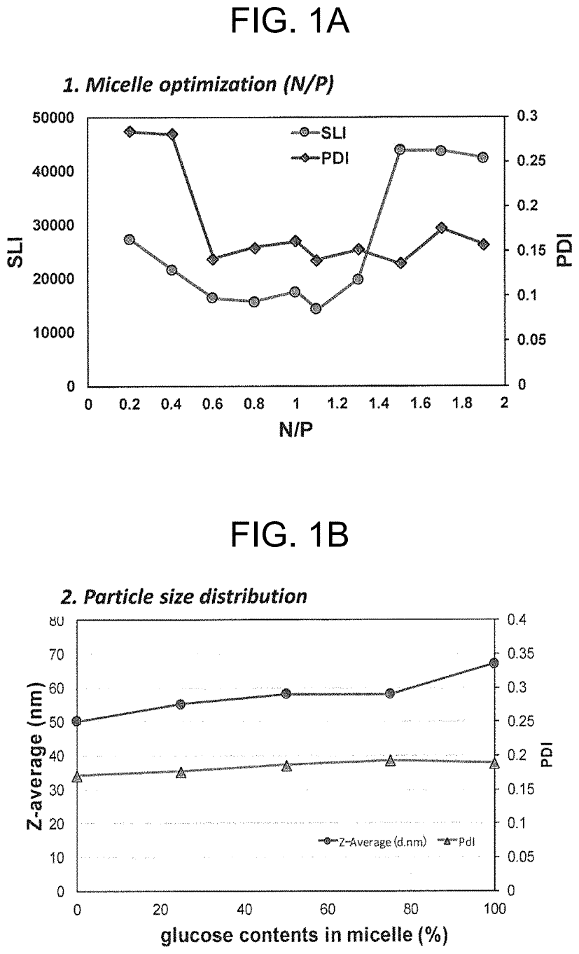

Glc(6)-PEG-PLys (DTBP/IM) was dissolved at 5 mg/mL in a 10 mM HEPES solution containing 100 mM dithiothreitol. ASO was dissolved in a 10 mM HEPES solution at a concentration of 30 μM. The solutions were mixed so as to ensure balanced electric charge, and the mixture was stirred and mixed for 15 seconds. Thereafter, the resulting liquid was put into a dialysis cassette (MWCO: 3,500, manufactured by Thermo-Fisher Scientific), and dialyzed for 2 days with 0.5% dimethyl sulfoxide (manufactured by Wako Pure Chemical Industries, Ltd.) and a 5 mM HEPES solution as an external solution. Thereafter, the external solution was replaced by a 5 mM HEPES solution, and dialysis was performed again for 2 days to obtain a Glc-PIC (ASO) micelle (hereinafter, also referred to as an “ASO micelle”) (see FIG. 1). The sequence of ASO was 5′-GGGTCagctgccaatGCTAG-3′ {here, uppercase characters correspond to LNA and lowercase characters correspond to DNA}. When a fluorescence label was added, the 3′-terminus of ASO was labeled.

A N/P ratio giving balanced electric charge was determined as follows. Specifically, for PIC (ASO), PEG-PLys was mixed with ASO at various N/P ratio (number of amidine groups/number of phosphate groups) values ranging from 0.2 to 1.9, and micelles were prepared in the same manner as described above. Thereafter, using Zetasizer, the particle size, the scattering light intensity (SLI) and the polydispersion index (PDI) were measured. The results were as shown in FIG. 1A. As shown in FIG. 1A, it was indicated that when the N/P ratio was 0.5 or less, the value of PDI was high, and micelles were not adequately formed. On the other hand, when the N/P ratio was 1.5 or more, SLI reached a plateau. This indicates that when the N/P ratio was 1.5, the cationic polymer and ASO electrically neutralized. Therefore, in the subsequent experiments, micelles were formed by mixing the cationic polymer and ASO in such a manner that the N/P ratio was 1.5.

### Example 2: Glucose Modification Ratio

Next, relationships between the glucose modification ratio and the particle formation, blood retentivity and accumulation on the brain were examined.

### (1) Relationships Between Glucose Modification Ratio and Particle Formation and Also Stability of Particles

Glc(6)-PEG-PLys (DTBP/IM) was mixed with methoxy-PEG-PLys (DTBP/IM) at a molar ratio of 100:0, 75:25, 50:50, 25:75 or 0:100 to adjust the glucose modification ratio (also referred to as a glucose content) to 100%, 75%, 50%, 25% or 0%, respectively. Using Zetasizer, the particle sizes (Z-average) and the polydispersion indices (PDI) of the obtained micelles having various glucose contents were measured. As shown in FIG. 1B, a PDI of less than 0.2 was obtained at all the glucose contents of micelles, and micelles were adequately formed. The particle size was about 50 to 70 nm, and the particle size increased as the glucose content became higher.

Further, the stability of the ASO micelle in vivo was evaluated. The stability was measured by fluorescence correlation spectroscopy (FCS). Micelles having glucose contents of 100% and 0% were prepared using Alexa 647-labeled ASO. The respective micelles were diluted with a physiological saline solution containing 10% FBS to prepare solutions with an ASO concentration of 5 nM to 5 μM, and the solutions were incubated at 37° C. for 1 hour. 100 μL of each of the samples was transferred to an 8-hole Lab-Tek chamber (Nalge Nunc Int. Corp., Rochester, N.Y.), and observed with Zeiss LSM 510 (Carl Zeiss, Germany) combined with a ConfoCor3 system. The Alexa 647 was excited with a He-Ne laser (633 nm) provided with a bandpass excitation light filter. The corresponding diffusion time was analyzed using Zeiss Confocor3 software. The results were as shown in FIG. 1C. As shown in FIG. 1C, there was no significant change in diffusion time at a concentration of 50 nM or more for any of the micelles. This result indicates that even in a severe environment in the blood, micelles can stably exist without desorbing.

### (2) Relationship Between Glucose Modification Ratio and Blood Retentivity in VIVO

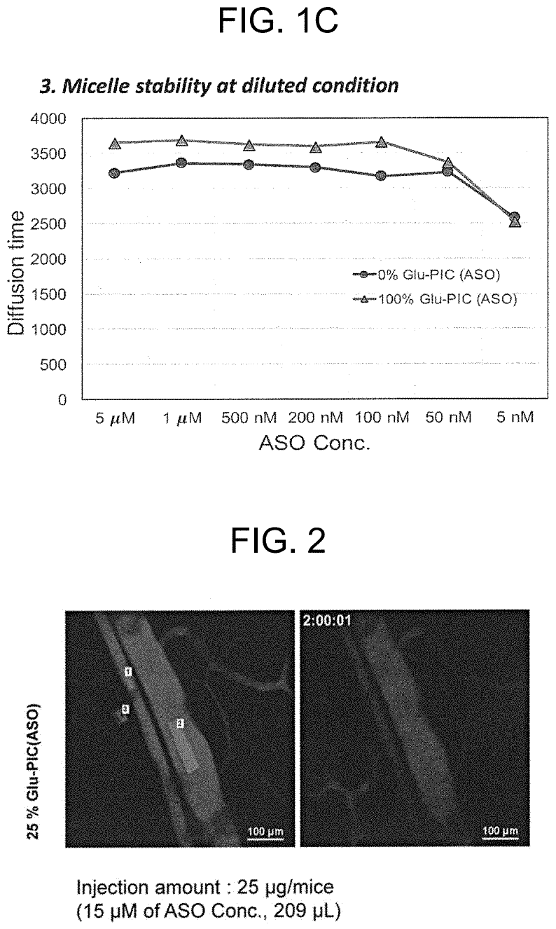

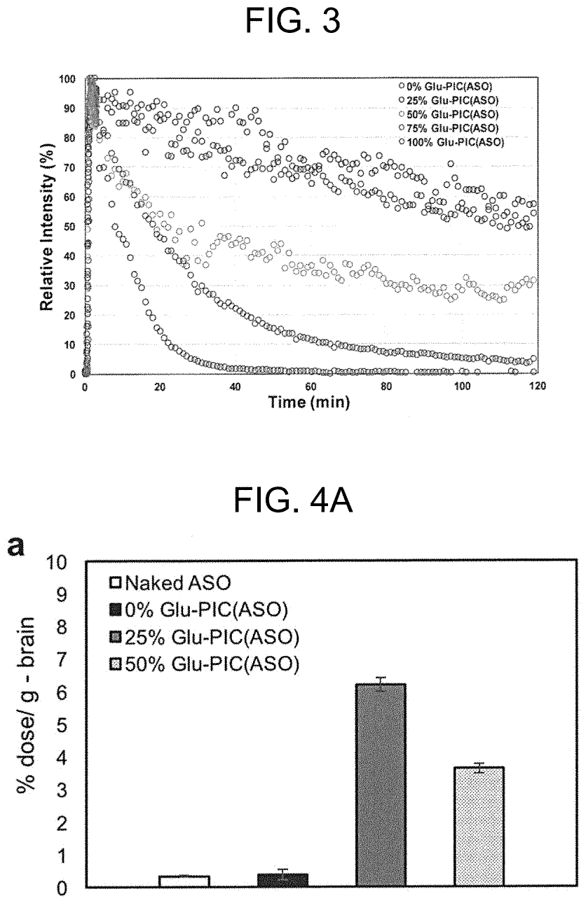

All animal experiments were conducted in conformity with the pertinent regulations of Tokyo University Animal Ethic Committee. Micelles containing Alexa 647-labeled ASO (22.5 μM) were prepared, and intravenously administered to mice (BALB/c, 6-week-old female mice). Thereafter, a time-dependent change in fluorescence intensity was observed with IVRT-CLSM (Nikon Corp., Tokyo Japan). The results were as shown in FIGS. 2 and 3.

As shown in FIG. 3, the highest blood retentivity was obtained when the glucose content was 0% or 25%. It was revealed that the blood retentivity decreased as the glucose content became higher.

FIG. 2 shows a fluorescence electron microscope image of a micelle, which contains 15 μM ASO and has a glucose content of 25%, in the blood vessel of the ear as a typical example. FIG. 2 shows a state in which fluorescence derived from Alexa 647 labelling ASO contained in the micelle declines in 2 hours.

### Example 3: Delivery of Micelle to Brain

In this Example, the delivery efficiency of ASO micelles to brain parenchyma was evaluated using the ASO micelles prepared as described above.

Glc-PIC (ASO) having a glucose content of 0%, 25% or 50% was prepared using Alexa 647-labeled ASO as described above. Normal mice (BALB/c, 6-week-old female mice, n=3) or Alzheimer's disease mice (BALB/c, 6-week-old female mice, n=3) were fasted for 24 hours to reduce the blood sugar level. Thereafter, PBS containing 200 μL of 20 (w/v) % glucose was intraperitoneally administered. 30 minutes after the administration, the mice were subjected to tail vein administration of Glc-PIC (ASO) containing 20 μg of ASO and a glucose content of 0%, 25% or 50% or naked ASO which was not encapsulated in the micelle.

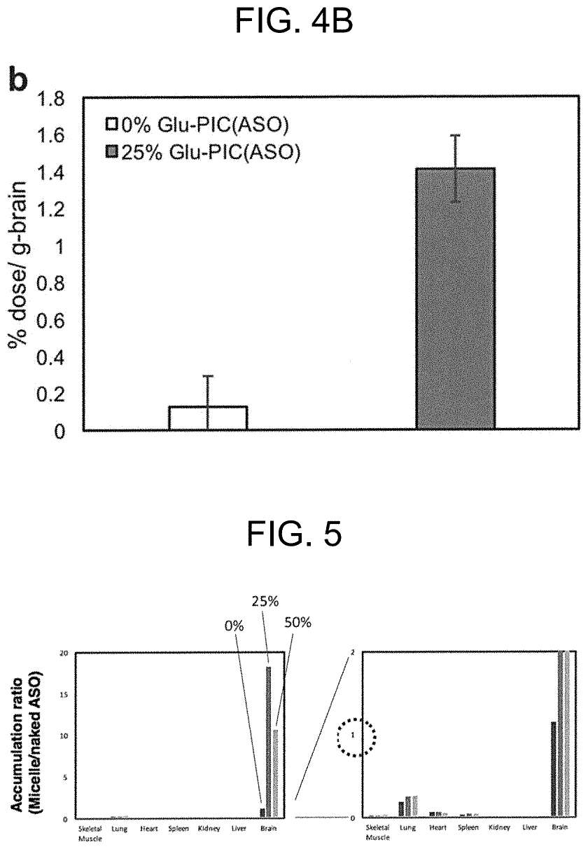

The mice were sacrificed, and brains were isolated. The isolated brain was washed with PBS to remove remaining blood. The weight of the brain was measured, and the brain tissues were finely cut. As a control, a sample obtained by mixing brain tissues with PBS or a micelle solution in an amount equivalent to the dose was used. The tissues were homogenized in a lysis buffer solution using a multi-bead shocker, and centrifuged at 2,500 rpm for 10 minutes. 100 μL of the supernatant was transferred to a 96-hole black plate, and the fluorescence intensity of each sample was measured using a TECAN microplate reader. The fluorescence intensity of brain tissues was shown as a relative fluorescence intensity against that of the control. The results were as shown in FIG. 4A.

As shown in FIG. 4A, there was little fluorescence derived from Alexa 647 in the brain tissue for naked ASO or Glc-PIC (ASO) having a glucose content of 0%, whereas for the micelle having a glucose content of 25% and the micelle having a glucose content of 50%, fluorescence was observed at 6.2% and 3.6%, respectively.

Accumulation of micelles in the brain of the Alzheimer's disease mouse was examined. As shown in FIG. 4B, the accumulation of the micelle having a glucose content of 25% in the brain was 11 times higher than the accumulation of the micelle having a glucose content of 0% in the brain. This result indicates that it may be possible to use Glc-PIC (ASO) as a drug delivery system for treatment of Alzheimer's disease.

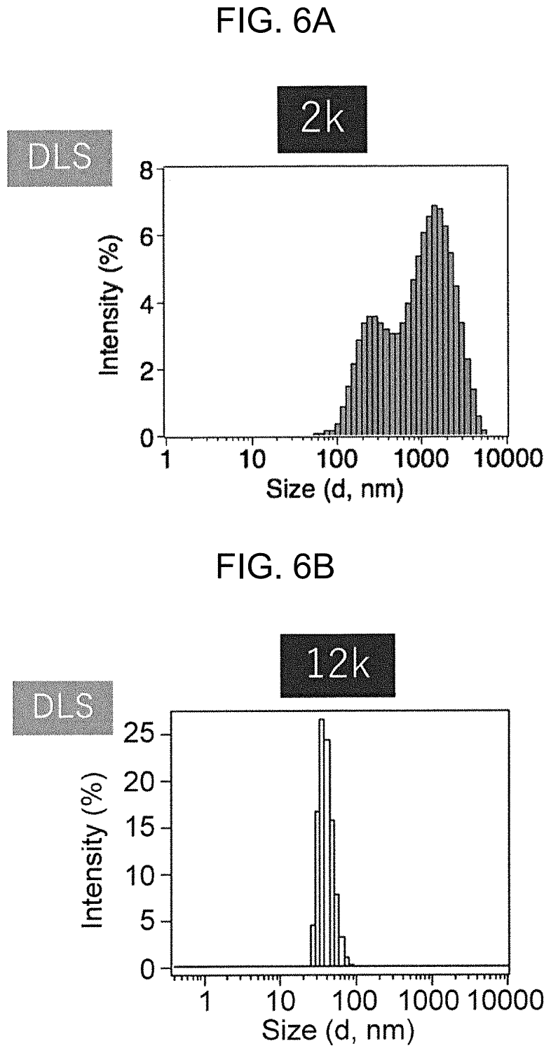

FIG. 5 shows the ratios of the accumulation of Glc-PIC (ASO) having a glucose content of 25%, in the brain and organs, to the accumulation of naked ASO in the brain and organs. This reveals that the accumulation of Glc-PIC (ASO) having a glucose content of 25% is lower than the accumulation of naked ASO in the liver and all other tissues, and is specifically high in the brain. In the figure, “0%” means that the glucose modification ratio is 0%, “25%” means that the glucose modification ratio is 25%, and “50%” means that the glucose modification ratio is 50%.

### Reference Example

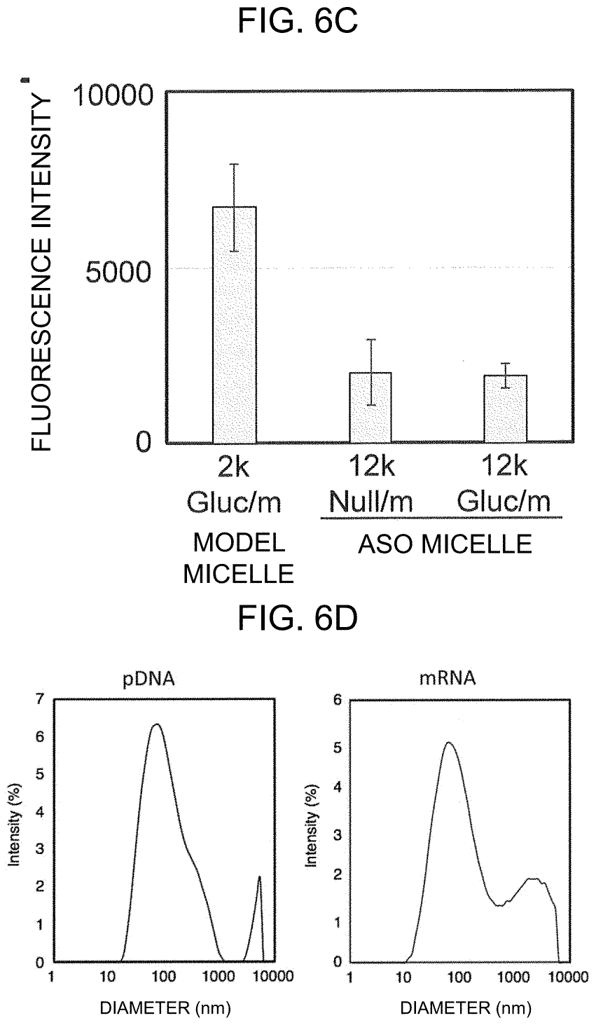

In this Reference Example, Glc-PIC (ASO) micelles were formed by the same method as described in Examples above except that the number average molecular weight of PEG was changed. More specifically, the micelle formation efficiency and the delivery efficiency to the brain are shown for a Glc-PIC (ASO) micelle in which the number average molecular weight of a PEG block is 2 kDa and a Glc-PIC (ASO) micelle in which the number average molecular weight of a PEG block is 12 kDa. The results were as shown in FIGS. 6A to 6C.

As shown in FIG. 6A, when the average molecular weight of PEG in the PEG block was 2 kDa, a micelle was formed, but the micelle had a bimodal particle size distribution, and the micelle formation efficiency was not high, whereas a micelle having a PEG block with a molecular weight of 12 kDa exhibited high micelle formation efficiency as shown in FIG. 6B. The micelle having a PEG block with a molecular weight of 12 kDa had a monodisperse particle size distribution. The same result was obtained irrespective of the type of DNA, RNA and an antibody.

As shown in FIG. 6C, the accumulation amount of micelles in the brain was 6.2% for the ASO micelle having a PEG block having an average molecular weight of 2 kDa (Gluc/m(2k)), whereas the accumulation amount of micelles in the brain was 0.2% for the ASO micelle having a PEG block having an average molecular weight of 12 kDa (Gluc/m(12k)). From this result, it was evident that the micelle having a PEG block having an average molecular weight of 2 kDa was advantageous in delivery efficiency of micelles to the brain. In this experiment, model micelles having a number average molecular weight of 2 kDa were prepared using Gluc-PEG-PAsp (2k-80), PEG-PAsp (2k-75) or PEG-DAP (2k-76) instead of using ASO {where DAP represents PEG-Pasp having a side chain modified with diaminopentane, 2k represents a number average molecular weight of PEG, and the following number represents a number average polymerization degree of an amino acid}.

Thus, when the number average molecular weight of the PEG block is 12 kDa, the micelle formation efficiency is high, but the accumulation efficiency in the brain is low, and when the number average molecular weight is 2 kDa, the accumulation efficiency in the brain is high, but the micelle formation efficiency is low.

In this Reference Example, using the cationic polymer in which the average molecular weight of PEG in the PEG block is 5 kDa, and plasmid DNA (pDNA) or mRNA, micelles were formed by the same method as the method for preparing ASO micelles as described in Examples above. Here, plasmid DNA having a nucleotide sequence of SEQ ID NO: 2 (4931-base length) was used as pDNA, and mRNA having a nucleotide sequence of SEQ ID NO: 3 (720-base length) encoding EGFP was used as mRNA. Examination of micelle formation efficiency revealed that as shown in FIG. 6D, either use of pDNA or use of mRNA as the anionic polymer produced a result different from the result obtained using ASO, that is, the obtained micelle was not monodisperse, and was poorer in micelle formation efficiency than the ASO micelle.

From this, the above-described result showing that the formation efficiency of Glc-PIC (ASO) including a PEG block having a number average molecular weight of 5 kDa is high and the accumulation efficiency of the Glc-PIC (ASO) in the brain is high as in Examples can be understood to be a result which depends on the number average molecular weight of PEG and the type of anionic polymer. That is, when the number average molecular weight of PEG is 2 kDa, the forming efficiency of Glc-PIC (ASO) is low, which is disadvantageous in terms of production cost and production efficiency, and when the number average molecular weight of PEG is 12 kDa, the delivery efficiency to the brain is low, which is disadvantageous. When PEG having a number average molecular weight intermediate between the above-mentioned number average molecular weights was used as in Examples, use of ASO as the anionic polymer led to much higher micelle formation efficiency as compared to pDNA and mRNA, and accordingly, the Glc-PIC (ASO) was excellent in both micelle formation efficiency and delivery efficiency to the brain.

1. A polyion complex micelle comprising an antisense oligonucleotide and a block copolymer of a PEG block having a number average molecular weight of 3 kDa to 10 kDa and a cationic polymer.

2. The polyion complex micelle according to claim 1, wherein a PEG-side end of the block copolymer of a PEG block having a number average molecular weight of 3 kDa to 10 kDa and a cationic polymer is modified with a molecule which binds to a cell surface or an extracellular matrix.

3. The polyion complex micelle according to claim 2, wherein the molecule which binds to a cell surface is a GLUT1 ligand.

4. The polyion complex micelle according to claim 3, wherein the GLUT1 ligand is glucose.

5. The polyion complex micelle according to claim 4, wherein 10 to 40 mol % of the PEG-side end of the block copolymer of a PEG block having a number average molecular weight of 3 kDa to 10 kDa and a cationic polymer is modified with glucose.

6. The polyion complex micelle according to claim 1, wherein the number average molecular weight of the PEG block is 4 kDa to 7 kDa.

7. A pharmaceutical composition comprising the polyion complex micelle according to claim 1.

8. A method for administering a pharmaceutical composition comprising the polyion complex micelle of claim 1 to a subject in accordance with a dosing regimen, the method comprising:
administering the pharmaceutical composition to a subject fasted or having hypoglycemia induced, and
inducing elevation of blood sugar level in the subject,
wherein the polyion complex micelle has an outer surface modified with a GLUT1 ligand.
